SEQUENCE LISTING (9 sequences)

SEQ ID NO: 1 - RNA, 17 nt, Human immunodeficiency virus type 1
ggagccaguagauccua

SEQ ID NO: 2 - RNA, 17 nt, Human immunodeficiency virus type 1
ggagccaguagauccua

SEQ ID NO: 3 - DNA, 38 bp, Artificial
  Rz2 ribozyme sequence
ttaggatcctgatgagtccgtgaggacgaaactggctc

SEQ ID NO: 4 - protein, 9 aa, Homo sapiens
ILKEPVHGV

SEQ ID NO: 5 - protein, 9 aa, Homo sapiens
SLYNTVATL

SEQ ID NO: 6 - protein, 12 aa, Artificial
  CD8 peptide
AAEGLDTQRFSG

SEQ ID NO: 7 - DNA, 41 bp, Artificial
  Sub G5-FD
caccaaaagagaactgcaatguttcaggacccacaggagcg

SEQ ID NO: 8 - DNA, 21 bp, Artificial
  5L1A primer
gagttctaccggcagtgcaaa

SEQ ID NO: 9 - DNA, 76 bp, Artificial
  3L1Dz5 primer
caccaaaagagaactgcaattcgttgtagctagcctttcaggacccacaggagcggcaagcaattcgttctgtatc

(19) United States
(12) Patent Application Publication
(10) Pub. No.: US 2006/0051866 A1
(43) Pub. Date: Mar. 9, 2006

(54) Means of producing and utilising a population of disease specific cytotoxic t-lymphocytes

(76) Inventors: Geoffrey Philip Symonds, Sydney (AU); Susan Margaret Pond, Sydney (AU); Gregory Charles Fanning, Sydney (AU)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/534,135

(22) PCT Filed: Nov. 7, 2003
(86) PCT No.: PCT/AU03/01476

§ 371 Date: May 6, 2005

Related U.S. Application Data
(60) Provisional application No. 60/424,514, filed on Nov. 7, 2002.

(51) Int. Cl. C12N 15/86; C12N 15/87; C12N 5/08

(52) U.S. Cl. 435/375; 435/455; 435/372; 435/456

(57) ABSTRACT

The present invention relates to the production of a population of virus-specific CTLs in which the population contains an anti-virus specific gene expression construct. This population can be combined with a population of CD4+ T lymphocytes or a population of CD34+ hematopoietic progenitor cells and one or more of these populations of cells can be delivered to an autologous virus-positive individual. The present invention also relates to therapeutic cell products and to methods of treating diseases, particularly infectious diseases.

### FIELD OF THE INVENTION

The present invention relates to cell and gene therapy, particularly as applied to hematopoietic cells of the Cytotoxic T Lymphocyte (CTL) class, either alone or in combination with exogenous gene-containing hematopoietic cells of the CD4+ T-lymphocyte class and/or the hematopoietic progenitor (HP) cell type. The invention relates to methods of producing these cell populations and to the delivery of the cells to subjects for therapeutic effect.

### BACKGROUND OF THE INVENTION

In recent years, research has been directed to developing therapies that use the patient's own immune system. One such approach is adoptive immunotherapy (Heslop et al, 1996; Walter et al, 1995). Adoptive immunotherapy aims to use the patient's cells to increase production of cytotoxic T lymphocytes (CTLs) to treat cancer or infection. This technique has shown promise as a potential clinical treatment regime for human patients (Heslop et al, 1996; Walter et al, 1995). The process works most effectively when the proper epitopes for presentation to the CTLs (this can be likened to an ex vivo “education” process of the CTLs) are known or can be identified. Adoptive immunotherapy preferably also requires the presence of antigen presenting cells (APCs) that are capable of expressing at least one disease-specific epitope. Current methods are now known for the efficient production of APCs and include, but are not limited to, International Publication PCT/US02/05748.

Other APC systems have been used to generate antigen-specific CTLs to a single epitope, including: 1) human dendritic cells (DC) pulsed with defined peptides; 2) peripheral blood mononuclear cells (PBMCs) which have been driven to lymphoblasts and pulsed with peptides; 3) lymphoblastoid cell lines (LCL) where the natural peptides are acid-stripped and loaded with the peptides of interest; 4) Drosophila cells engineered to express empty class I molecules; and Mouse 3T3 cells transfected with human class I and co-stimulatory molecules (Latouche and Sadelain, 2000).

International publication PCT/US02/05748 demonstrates that CTLs can be produced to exhibit peptide-specificity to several HLA-A2.1-restricted peptides from melanoma-associated antigen. These CTLs have been isolated from leukopheresis samples and presented in vitro with melanoma antigenic peptide epitopes using Drosophila cells as the non-natural antigen-presenting cells (nnAPCs). The CTLs are expanded over a period of 20 or 21 days and incubated with autologous monocytes APCs loaded with the melanoma antigenic epitopes in the presence of Interleukin-2 (IL-2) and Interleukin-7 (IL-7). This treatment is followed by non-specific expansion using OKT3 for 10 days and the product is infused into patients.

The introduction of a therapeutic gene into hematopoietic cells is an attractive possibility for protecting CTLs from viral infection. For example, CTLs are particularly sensitive to HIV infection. Therapeutic genes have been introduced into CD34+ pluripotent hematopoietic progenitor cells to target HIV gene expression. Hematopoietic progenitor cells may be readily separated from more mature hematopoietic cells by using the CD34+ antigen. The antigen is a membrane-bound 115 Kd molecule present on cells but absent on mature hematopoietic cells. CD34+ cells can give rise to multi-lineage colony forming cells, (Baum et al. 1992) and are capable of relatively rapidly (3-6 months) reconstituting lymphoid (CD4+ and CD8+ T-lymphocytes) and myeloid (monocyte/macrophages) hematopoiesis through a series of intermediate cells of increasing maturity (Levinsky 1989; Schwartzberg et al. 1992). Like CD34+ cells and CD4+ cells, CD8+ lymphocytes may be candidates for gene therapy ex vivo; however the ability of transduced CD8+ cells to target HIV nucleic acid in the body is not known. CD4+ and CD8+ T-lymphocytes may be separated from other cell types by means of the CD4 and CD8 receptor respectively. Cytotoxic T-lymphocytes CTLs are a sub-set of CD8+ T-lymphocytes that are involved in cellular response to infections and malignancies (Janeway et al, 1999). CD4+ T lymphocytes are involved in T-lymphocyte helper function for B cells (antibody production—Janeway et al, 1999) and helper function for CD8+ T-lymphocytes.

Part of the reason for the decline in CD8+ CTLs in later stage HIV/AIDS is the loss of CD4+ T-lymphocytes leading to lack of helper function (Levy, 1994). Key cells in terms of HIV/AIDS infection are the CD4+ and CD8+ T-lymphocytes and monocyte/macrophages. Cells of these types, isolated and then re-infused into autologous individuals, have been shown to engraft and function normally (Schindhelm and Nordon, 1999).

Ribozymes are small catalytic RNA moieties capable of cleaving specific RNA target molecules. Ribozymes have been used to target a number of nucleic acid sequences. For example, ribozymes directed against HIV-1 can interfere with HIV-1 replication by interfering in several steps in the HIV-1 life cycle including the production of genomic viral RNA in recently infected cells (prior to reverse transcription) and the production of viral RNA transcribed from the provirus before translation or prior to genomic RNA packaging (Sarver et al. 1990; Sun et al. 1996; Sun et al. 1998). Theoretically, ribozymes are more effective than antisense in their ability to inhibit gene expression because ribozymes are catalytic molecules that not only bind to their target but cleave their target. Moreover, ribozymes can cleave multiple RNA substrate molecules (Sarver et al. 1990; Sun et al. 1996).

The requirements for cleavage by a ribozyme are an accessible region of RNA and, in the case of the hammerhead ribozyme, the need for a GUX target motif or, in certain cases, NUX may suffice (where N is any ribonucleotide and X is A, C or U ribonucleotides). In contrast to the therapeutic use of proteins such as transdominant rev or intracellular antibodies, catalytic RNAs are unlikely to provoke an immune response that leads to the elimination of cells that contain the exogenous gene.

A number of studies have demonstrated ribozyme cleavage activity in test tube reactions, and protective effects in tissue culture systems against laboratory and clinical isolates of HIV-1 (Sarver et al. 1990; Sun et al. 1998; Wang et al. 1998). These studies used either hammerhead or hairpin ribozymes. For example, a hammerhead ribozyme directed against a highly conserved region of the tat gene is provided in FIG. 1 as Rz2. The tat gene is essential for HIV-1 replication in that it encodes and produces the Tat protein. The Tat protein is a transcriptional activator of integrated HIV-1 provirus. The Rz2 complementary hybridizing and target sequences comprise nucleotides 5833-5849 (GGAGCCA GUA GAUCCUA) of reference strain HIV-HXB2 (Genbank accession number K03455) or nucleotides 5865 to 5882 (GGAGCCA GUA GAUCCUA) of HIV IIIB (Genbank accession number X01762). The Rz2 ribozyme sequence 5′-TTA GGA TCC TGA TGA GTC CGT GAG GAC GAA ACT GGC TC-3′ has been inserted as DNA into the 3′ untranslated region of the neo^{R} gene within the plasmid pLNL6, which contains the replication-incompetent retroviral vector LNL6 (Genbank accession number M63653) to generate a new virus, RRz2. The ribozyme sequence has been expressed as a neo^{R}-ribozyme fusion transcript from the Moloney Murine Leukemia Virus (MoMLV) Long Terminal Repeat (LTR) in RRz2 as disclosed previously (Sun, L.-Q. et al. (1995) Proc. Natl. Acad. Sci. USA, 92, 7272-7276; Sun, L.-Q., et al (1995) Nuc. Acids Res., 23, 2909-2913; and Sun, L-Q., et al. (1998) Methods in Molecular Medicine, Therapeutic Applications of Ribozymes (ed K J Scanlon) Humana Press USA, p 51-64.

The ribozyme gene transfer product RRz2 has been used in two Phase I Clinical Trials. In each trial, approximately half of each relevant cell population (CD4+ or CD34+HP cells) was transduced with RRz2 and the other half with the control vector LNL6, following which the cells were mixed and reinfused (see for example, Amado, R. et al. (1999) Human Gene Therapy, 10:2255-2270).

In the first trial, involving 4 pairs of genetically identical twins discordant for HIV infection, the RRz2 construct was introduced into a population of CD4+ T-lymphocytes (from the HIV negative twin) ex vivo and these cells (in a background of non-gene containing T-lymphocytes) were infused into the corresponding HIV positive twin. Subsequent to infusion, ribozyme construct presence and expression in mature lymphoid cells was seen for at least 4 years (the latest time point examined). This Phase I study has shown that this approach is technically feasible and safe.

In a second Phase I study, the RRz2 construct was introduced into CD34+ progenitor cells ex vivo. The infusion of RRz2-containing CD34±HP cells gave rise to RRz2-containing peripheral blood cells including CD4+ and CD8+ T-lymphocytes, as determined by sensitive PCR methods. Ribozyme construct presence and expression was seen in both mature lymphoid and myeloid cells for up to 3.5 years (the latest time point examined). The greater dose of transduced CD34+ hematopoietic progenitor cells given, the greater the persistence of the ribozyme-containing cells. In addition evidence was obtained for preferential survival of the RRz2 containing T-lymphocytes over the control vector containing T-lymphocytes, indicating that the ribozyme renders the T-lymphocytes at least partially protected from HIV-1 infection and replication. These two Phase I studies have shown that the approach, namely the introduction and persistence of gene-therapeutic containing cells, is technically feasible.

Anti-retroviral drugs are used in the treatment of HIV/AIDS. These drugs predominantly target the reverse transcriptase and protease steps of the HIV life-cycle. As of November 2002 there are of the order of 15 drugs that are in clinical use for the treatment of HIV/AIDS (Guidelines for the Use of Antiretroviral Agents in HIV-Infected Adults and Adolescents, 2002; http://www.hivatis.org). Generally two nucleoside and one non-nucleoside reverse transcriptase inhibitors or two nucleoside reverse transcriptase and one protease inhibitor are the initial drug combinations of choice. It is generally accepted that such drug treatments should not commence until there is a clinical need; the reasoning being that the drugs drive viral resistance and have side effects that can become severe. In addition it is very difficult for the patient to take all drugs at all required times (i.e. to be 100% compliant). The presently available evidence indicates that the drugs will be required for life and thus it is thought that the less the drugs need to be given, the less the chance of driving viral resistance and causing more and more pronounced side effects. Thus, methods that are able to reduce the need or the duration of antiviral drugs are desired.

It is also thought that the ability of the HIV-specific CTL population to impact disease progression decreases with time because the CTLs are destroyed by the disease process and, when the drugs are used, there is less HIV replication for them to respond to. In part, this is due to the lack of production of new HIV-specific CTLs during maturation in the absence of viral epitopes. Therefore, there is a need for therapies that enhance CTL survival.

In addition, for all of the above reasons (driving HIV resistance, drug side effects, difficulty with 100% patient compliance to the drug regimens at all times, the suppression of a HIV-specific CTL response), there is a body of evidence which supports the notion of the withdrawal of the antiretroviral drugs for periods of time (hereinafter referred to as “treatment interruptions”, see guidelines for the Use of Antiretroviral Agents in HIV-Infected Adults and Adolescents, 2002; http://www.hivatis.org). Such treatment interruptions may be relatively short (several weeks) or of more prolonged duration (several months) and the safety of the Treatment Interruptions is monitored by assessment of viral load and CD4+ T-lymphocyte count, along with other safety measures.

### SUMMARY OF THE INVENTION

In a first aspect of the present invention, there is provided a method for producing a cytotoxic T-lymphocyte population primed for virus-specific CTL activity comprising the steps of:

(a) preparing non-naturally occurring antigen-presenting cells(nnAPC) which present at least one virus-specific antigen;

(b) harvesting a population of white blood cells from a subject;

(c) incubating a population of CD8+ cells obtained from the white blood cells in step (b) with the nnAPC cells; and

(d) treating the CD8+ cells with one or more supportive cytokines.

In a second aspect of the present invention, there is provided a method for producing a cytotoxic T-lymphocyte population transduced with virus-inhibiting nucleic acid and primed for virus-specific CTL activity comprising the steps of:

(a) preparing a non-naturally occurring antigen presenting cell line (nnAPC) which presents at least one virus specific antigen;

(b) harvesting CD8⁺ cells from a subject;

(c) incubating the CD8⁺ cells with the nnAPC cell line;

(d) adding Interleukin-2 (IL-2) and Interleukin-7 (IL-7) to the CD8⁺ cells after step (c);

(e) introducing at least one virus-inhibiting nucleic acid into the CD8⁺ cells wherein the virus inhibiting nucleic acid is expressed; and

(f) incubating the CD8⁺ cells with non-proliferating peripheral blood mononuclear cell-derived adherent cells and wherein the adherent cells present at least one of the same virus-specific antigens of step (a).

The present invention relates in one embodiment, to the use of cytotoxic T-lymphocytes (CTLs) that have been specifically exposed, and are sensitized, to viral disease specific peptide epitopes ex vivo for the purpose of eradicating diseased cells when those CTLs are re-infused into a patient. Further, the invention relates in another embodiment, to genetically modifying at least a percentage of the sensitized CTLs so that they are protected or are resistant to viral disease. In yet another form, the first mentioned embodiment may be used alone or in combination with the second mentioned embodiment above. In still another preferred embodiment, the virus-specific CTLs, which may comprise a percentage of gene modified CTLs, can be used alone or in combination with other hematopoietic cells, a proportion of which are gene-modified with disease-specific gene(s).

The viral disease-specific cytotoxic T lymphocytes (CTLs) can be used for the treatment of infectious diseases, and further transduction of the cells with a disease protective genetic agent may further help in this treatment. These transduced CTLs can be used alone or in combination with other cell populations, namely CD4+ and CD34+ cell populations that have been transduced with a therapeutic (or marker) gene construct for effect on disease. This approach will be useful for therapy of HIV-1 and for other cell/gene therapies in which protected blood cell populations are required. The invention further relates to the use of hematopoietic cells, preferably transduced ex vivo (for example, with an anti-HIV-1 gene therapeutic), to be introduced into a recipient patient alone or in combination with other such hematopoietic cells in sufficient number to produce a chimeric hematopoietic system comprising enough disease specific (and in one embodiment anti-HIV agent-containing hematopoietic cells) targeting moieties to have a therapeutic effect.

The anti-disease CTLs (with or without the anti-disease gene), may be used alone or in combination with a proportion of anti-disease gene-containing CD4+ T-lymphocytes and/or a proportion of anti-disease gene-containing hematopoietic progenitor cells, the latter population being able to produce a proportion of anti-disease gene-containing myeloid (monocyte/macrophages) and lymphoid cells (including CD8+ and CD4+ T-lymphocytes), such that the cell population(s) impact on disease (e.g., HIV-1 infection and disease) progression.

In another aspect the present invention provides a therapeutic cell product comprising a cytotoxic T-lymphocyte population primed for virus-specific CTL activity produced according to the method of the first aspect.

In another aspect, the present invention provides a therapeutic cell product comprising a cytotoxic T-lymphocyte population transduced with virus-inhibiting nucleic acid and primed for virus-specific CTL activity produced according to the method of the second aspect.

In still another aspect of the present invention there is provided a method of treating a subject with an infectious disease, the method comprising:

(a) preparing a non-naturally occurring antigen-presenting cells (nnAPC) which present at least one virus-specific antigen;

(b) harvesting a population of white blood cells from the subject;

(c) incubating a population of CD8+ cells obtained from the white blood cells in step (b) with the nnAPC cells;

(d) treating the CD8⁺ cells with one or more supportive cytokines; and

(e) introducing the CD8+ cells from step (d) into the subject.

In one preferred embodiment of the method of treatment aspect, the subject has more than one infectious disease, and at least one virus-specific antigenic peptide specific for each infectious disease is utilised in step (a).

The virus inhibiting nucleic acid utilised in a preferred method of the invention may for instance be specific for a disease selected from the group consisting of Human papilloma virus, Cytomegalovirus, Epstein Barr Virus, Hepatitis A, Hepatitis B, Hepatitis C, Hepatitis D, Hepatitis E, Measles, Mumps, Polio, Rubella, Influenza, Yellow Fever, Japanese Encephalitis, Dengue, Rabies, Rotavirus, Varicella Zoster, Chikungunya Rift Valley Fever, Respiratory Syncitial Virus, Herpes Simplex, Coronaviruses, Marburg, Ebola, California Encephalitis Virus, JC Virus, Lymphocytic Choriomeningitis Virus, Parvovirus, Rhinovirus, Smallpox, HTLV-1, HTLV-2, and HIV.

The method of treatment aspect may also comprise incubating a population of CD4+ T lymphocytes obtained from the white blood cells in step (b), and/or a population of CD34+ haematopoietic progenitor cells with the nnAPC cells prior to introducing the CD4+ T lymphocytes and/or CD34+ progenitor cells into the subject.

In some such embodiments, one or more supportive cytokines are added to the CD4+ T lymphocytes and/or CD34+ haematopoietic progenitor cells. Moreover, a virus-inhibiting nudeic acid may be introduced into the population of CD4+ T lymphocytes such that the virus inhibiting nucleic acid is expressed in the lymphocytes, and/or into the population of CD34+ haematopoietic progenitor cells such that the virus inhibiting nudeic acid is expressed in the progenitor cells, prior to the CD4+ T lymphocytes and/or CD34+ progenitor cells being introduced into the subject.

In addition, treatment interruptions may be used to assist the administered CTL's (alone or in combination with other cell types) to impact on disease.

The supportive cytokines added to the CD8+ cells may be selected from the group consisting of IL-2, IL-4, IL-7, IL-15 and IL-21.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All references cited herein are incorporated by reference into this specification in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention before the priority date of each claim of this application.

### BRIEF DESCRIPTION OF THE ACCOMPANYING FIGURES

FIG. 1 provides an illustration of the location of a ribozyme target site within the HIV-1 genome. Panel A provides a schematic diagram of the HIV-1 genome showing location of replicative, regulatory and accessory genes; Panel B provides the ribozyme sequence together with the complementary target and hybridizing sequence within the tatgene. The target GUA cleavage site is circled; and C provides the location of the GUA target sequence in the genes encoding Tat and Vpr proteins.

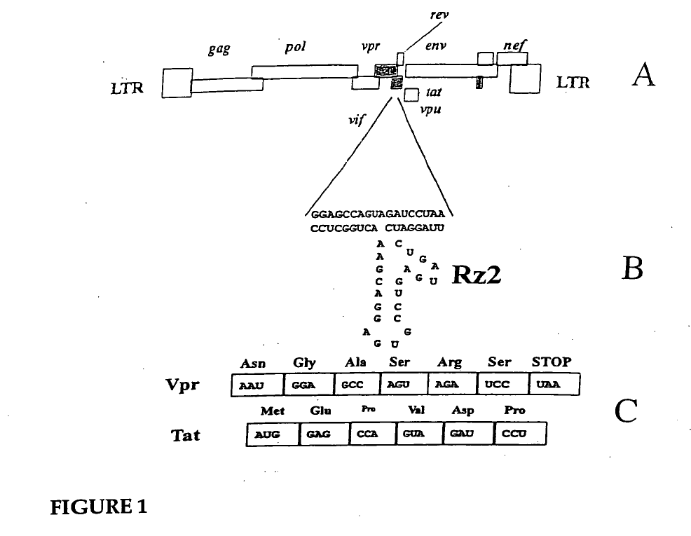

FIG. 2 provides an illustration of the various major steps of the method of this invention.

FIG. 3 provides an illustration of the antigen-presenting method involved in FIG. 2 above. Apheresis yields peripheral blood mononuclear cells from which CD8+ T-lymphocytes and monocytes are isolated.

FIG. 4 provides an illustration of the ontogeny of the various cell types—CD8+, CD4+, CD34+.

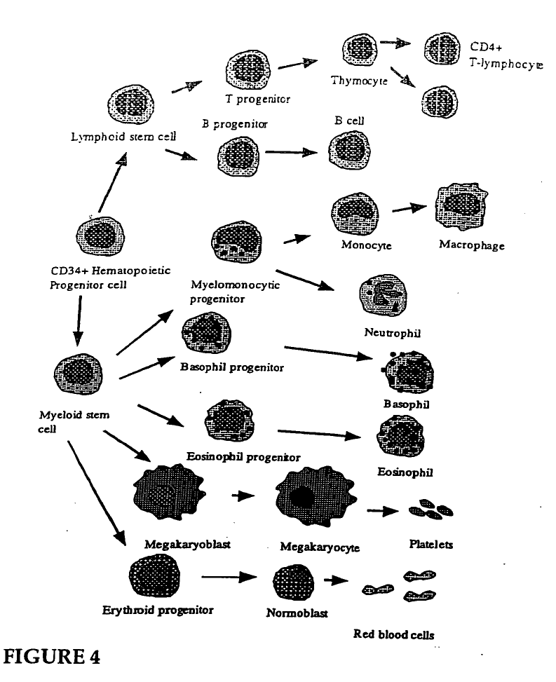

### DETAILED DESCRIPTION OF THE PREFERRED EMBODIMENTS OF INVENTION

Following is a list of abbreviations and definitions used in the present specification.

### Abbreviations

AIM-V The AIM-V medium for cell culture
APC Antigen-presenting cells
CD8⁺ CD8⁺ T cells
CD34+ cells cells, which have the CD34+ antigen on their surface; a subset of hematopoietic progenitor cells.
Cell purity the percentage of cells in any population positive for the requisite antigen.
CTL Cytotoxic T lymphocyte; a sub-set of CD8+ T-lymphocytes
DzyNA a method for real-time PCR detection and quantification of DNA or RNA such as that described in U.S. Pat. No. 6,140,055 and U.S. Pat. No. No. 6,201,113.
E Effector
Fas Also known as CD95, epitope on T cells
HP cell Hematopoietic Progenitor Cell; a pluripotential cell that in vivo continuously gives rise to all of the various lineages of the hematopoietic system.
ICAM Intercellular adhesion molecule
IL Interleukin
LAK Lymphokine-activated killer cells
LFA Lymphocyte function antigens
LNL6 a murine retroviral vector, derived from Moloney Murine Leukemia Virus, that has the replicative genes deleted and the neomycin phosphotransferase (neo^{r}) gene inserted. The vector is based on the retroviral plasmid, pLNL6, which contains the replication-incompetent retroviral vector LNL6 (Genbank accession number M63653).
MHC Major histocompatibility complex
nnAPC non-naturally occurring antigen-presenting cell
NP Nuclear protein
PBMC Peripheral blood mononuclear cell
PBS Phosphate-buffered saline
PCR Polymerase chain reaction
RPMI Roswell Park Memorial Institute
Rz2 anti-HIV hammerhead ribozyme targeted to a highly conserved region of the tatgene; the Rz2 ribozyme sequence is 5′-TTA GGA TCC TGA TGA GTC CGT GAG GAC GAA ACT GGC TC-3′.
RRz2 retroviral vector consisting of LNL6 with Rz2 inserted into the 3′ untranslated region of neo^{r}.
RWJPRI The R.W. Johnson Pharmaceutical Research Institute
T Target
TCR T cell antigen receptor
TIL Tumor-infiltrating lymphocytes
Transduction the introduction of a gene into a cell and the consequent expression of that gene in that cell.
VCM Virus containing medium

Following is a list of abbreviations used in the present specification for various peptide epitopes. The individual amino acid residues are identified according to a single letter code that is readily known and used by those of ordinary skill in the art.

Abbreviations

| Amino Acid | 3-Letter | 1-Letter |
|---|---|---|
| alanine | ala | A |
| valine | val | V |
| leucine | leu | L |
| isoleucine | ile | I |
| proline | pro | P |
| phenylalanine | phe | F |
| trytophan | tyr | W |
| methionine | met | M |
| glycine | gly | G |
| serine | ser | S |
| threonine | thr | T |
| cysteine | cys | C |
| tyrosine | tyr | Y |
| asparagine | asn | N |
| glutamine | gln | Q |
| aspartic acid | asp | D |
| glutamic acid | glu | E |
| lysine | lys | K |
| arginine | arg | R |
| histidine | his | H |

As used herein, the term “ex vivo ” or “ex vivo therapy” refers to a therapy where biological materials, typically cells, are obtained from a patient or a suitable alternate source, such as, a suitable donor, and are modified, such that the modified cells can be used to treat a pathological condition which will be improved by the long-term or constant delivery of the therapeutic benefit produced by the modified cells. Treatment includes the re-introduction of the modified biological materials, obtained from either the patient or from the alternate source, into the patient. A benefit of ex vivo therapy is the ability to provide the patient the benefit of the treatment, without exposing the patient to undesired collateral effects from the treatment. For example, high doses of cytokines are often administered to patients with cancer or viral infections to stimulate expansion of the patient's CTLs. However, cytokines often cause the onset of influenza like symptoms in the patients. In an ex vivo procedure, cytokines are used to stimulate expansion of the CTLs outside of the patient's body, and the patient is spared the exposure and the consequent side effects of the cytokines. Alternatively under suitable situations, or conditions, where appropriate and where the subject can derive benefit, the subject can be treated concurrently with low level dosages of cytokines, such as, for example, IL-2. The effect of the IL-2 is to enhance antigen specific CTL persistence.

As used herein, the term “major histocompatibility complex” or “MHC” is a generic designation meant to encompass the histo-compatibility antigen systems described in different species including the human leucocyte antigens (HLA).

As used herein, the terms “epitope,” “peptide epitope,” “antigenic peptide” and “immunogenic peptide” refers to a peptide derived from an antigen capable of causing a cellular immune response in a mammal. Such peptides may also be reactive with antibodies from an animal immunized with the peptides. Such peptides may be about five to twenty amino acids in length preferably about eight to fifteen amino acids in length, and most preferably about nine to ten amino acids in length.

As used herein, the term “Interleukin 2 (IL-2)” refers to a cytokine which stimulates the immune system and which exerts its biological effects following binding to specific receptors on the surface of target cells. IL-2 has many biological effects, for example, it is known to induce the stimulation of activated B and T cells (including cytotoxic T cells), natural killer (NK) cells, and lymphokine activated killer (LAK) cells. IL-2 may be obtained as a prescription drug, for example, PROLEUKIN®, manufactured by Chiron Corporation (Emeryville, Calif.). IL-2 may be prepared from various sources and by different methods, as disclosed in numerous U.S. patents. These patents include, but are not limited to, the preparation of IL-2 from T cells, such as from hybrid murine T cell lines or malignant human T cell lines, as disclosed in U.S. Pat. Nos. 4,407,945, 4,473,642, and 4,401,756, respectively, and the preparation of recombinant human IL-2 as disclosed in U.S. Pat. Nos. 4,992,367, 4,407,945, and 4,473,642.

The present invention is directed at obtaining and preparing the therapeutic dose of an enriched pool of disease-specific CD8+ Cytotoxic T Lymphocytes (CTLs) a proportion of which, in one embodiment, are transduced with a therapeutic gene, for delivery to the patient. The therapeutic gene is preferably a gene or gene construct capable of limiting the dissemination of an infectious disease, and preferably a viral disease. These CTLs will be used alone or in combination with gene-containing CD4+ T-lymphocytes and/or gene-containing CD34+ hematopoietic progenitor cells. While the invention is exemplified with reference to the treatment of HIV, the invention is not limited thereto and methods described herein may be used in the treatment of other viral infections.

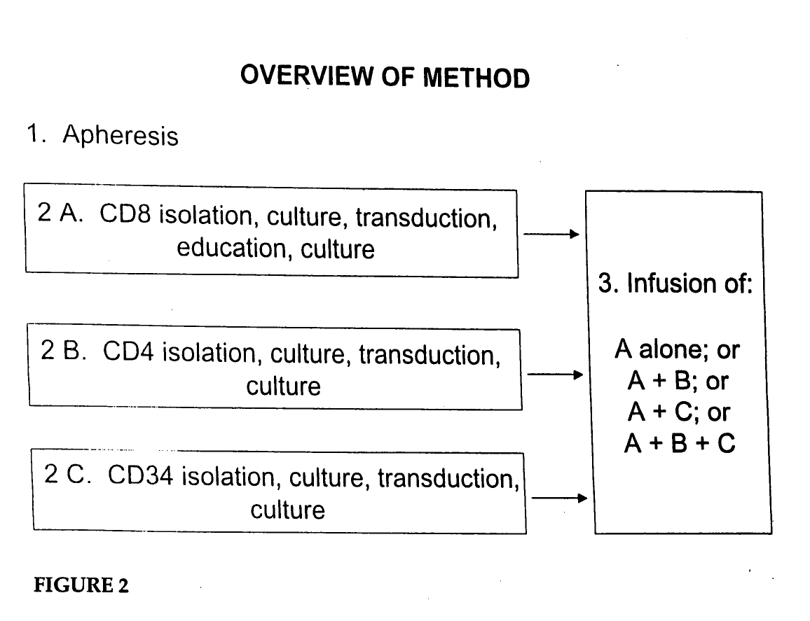

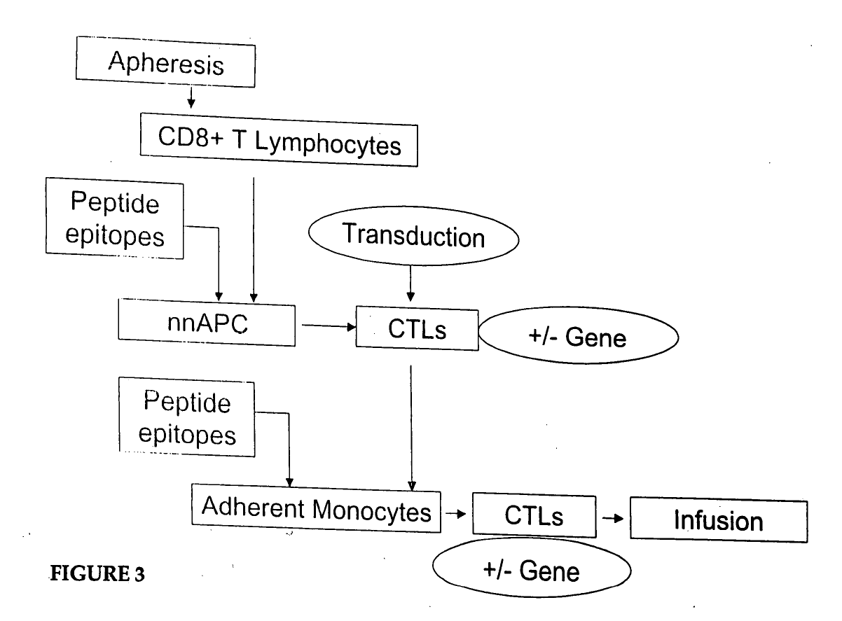

A preferred outline of the present invention is provided in FIGS. 2 and 3 and includes apheresis of a patient's blood to obtain peripheral blood mononuclear cells, either with or without G-CSF mobilization (required if CD34+ hematopoietic progenitor cells are required). CD8+, CD4+, and/or CD34+ T lymphocytes are separately isolated, cultured, optionally transduced, exposed to peptide, cultured, harvested and infused.

The invention contemplates that CD8⁺ cells, CD8+ cells in combination with CD4+ cells, CD8+ cells in combination with CD34+ cells or a combination of all three cell types can be infused back to the patient.

As a first step for practicing the methods of this invention, non-naturally occurring antigen-presenting cells (nnAPC) are prepared. The nnAPC are capable of being loaded simultaneously with at least one and up to fifteen different exogenous peptides where each peptide is preferably at least eight and more preferably eight to ten amino acids in length, and are capable of presenting the peptide molecules on the surface of their cells.

The peptides used to load the antigen presenting cells are associated with infectious diseases and preferably are associated with viral disease. A variety of peptides can be used, but preferably the peptides are known to stimulate cytotoxic T cell responses. Peptides, including viral-derived peptides, that can stimulate cytotoxic responses, have been identified in the art. In addition there are methods and assays that are known to determine which peptide fragments can stimulate cytotoxic T cell responses and can therefore be used to identify other peptides that can be used in this invention. The examples employ exemplary peptides derived from HIV protein to stimulate an HIV-specific cytotoxic T cell response.

Cytotoxic T lymphocytes (CTLs) are one of the natural barriers to viral infection. In humans these cells destroy virus-containing cells, thereby reducing viral load. In Human Immunodeficiency Virus (HIV) infection, for example, one of the major obstacles to clearing infection is that the virus decreases the production of the CTLs, in part by infecting and destroying this cell population. Methods described herein can provide a population of disease-specific CTLs with or without gene modification. The gene modification should mean that the disease-specific CTLs are not subject to disease induced depletion, thereby acting to ameliorate disease. The CTL population (with or without gene modification thereof) can be used alone or in combination with other gene-modified cells such as CD4+ T lymphocytes or CD34+ hematopoietic progenitor cells.

The CD8+ T-lymphocytes are first incubated with disease-specific peptide epitopes in the presence of nnAPCs and cultured for a further period of time. In a preferred embodiment, the CD8⁺ cells are incubated with the nnAPC cell line for about six to seven days. Supportive cytokines such as Interleukin-2 (IL-2) and Interleukin-7 (IL-7) can be added to the media 4 to 5 days after stimulation with the nnAPCs. In a preferred embodiment, 20 U/ml IL-2 and 30 U/ml of IL-7 are added to the media.

In another embodiment of the present invention the nnAPC that express the peptides are further modified to express other peptides or polypeptides that can enhance the treatment of the subject. For example in addition to presenting peptides associated with the disease or disease condition being treated, the nnAPC can express polypeptides associated with accessory molecules such as, lymphocyte function antigens (LFA-1, LFA-2 and LFA-3), intercellular adhesion molecule 1 (ICAM-1), and/or T-cell co-stimulatory factors (CD2, CD28, B7), in order to enhance cell-cell adhesion or transduce additional cell activation signals.

Following incubation with the nnAPC cells, the CD8+ cell population is transduced with an infectious disease protective gene therapeutic. Preferably the infectious disease protective gene therapeutic is introduced such that it is expressed in not only the transduced cells, but also the progeny of the transduced cells. The infectious disease protective gene therapeutic comprises nucleic acid and methods for introducing nucleic acid in a variety of forms into cells and is well known in the art. Preferably the infectious disease protective gene therapeutic is directed to viral disease. In one embodiment, the therapeutic is a ribozyme. Those skilled in the art will recognize that there are a variety of other types of gene therapeutics known in the art.

As one example of this invention CD8+ CTLs are transduced with an anti-HIV gene construct, RRz2, to produce a therapeutic effect by decreasing viral load and increasing CD4+ cell count as a result of the HIV directed CTL activity of the cells containing the therapeutic gene. These cells are specifically directed to HIV infected cells to destroy them yet where the CTLs, and optionally CD4+ cells, are themselves protected from the cytopathic effect of HIV by virtue of the presence of the RRz2 gene expression construct.

A number of investigators have proposed and tested a variety of gene therapy approaches using novel anti-Human Immunodeficiency Virus 1 (HIV-1) agents in tissue culture. These approaches include intracellular expression of transdominant proteins (Smythe et al. 1994), intracellular antibodies (Marasco et al. 1998), antisense ribonucleic acid (RNA) (Sczakiel et al. 1991), viral decoys (Kohn et al. 1999), and catalytic ribozymes (Sarver et al. 1990; Sun et al. 1996).

In a preferred next step for practicing the methods of this invention, the CD8± derived, CTL enriched population, is further incubated with the same disease-specific peptide epitopes in the presence of an adherent population of autologous monocytes which were produced following the isolation of the adherent cell population from the peripheral blood mononuclear cells. The CD8⁺ cells are incubated with peptide-loaded adherent cells for about six to seven days by: mixing adherent cells—obtained from the peripheral blood mononuclear cell population, most preferably adherent monocytes from the CD8+ depleted peripheral blood collected from said subject or a suitable donor with about 10 to 50 μg/ml of each peptide of interest.

Peripheral blood monocytes are isolated from the peripheral blood mononuclear cell population following apheresis. In one embodiment, the peripheral blood monocyte suspension is preferably irradiated with a sufficient dose of γ-radiation necessary (to prevent further cell proliferation of the PBMCs while maintaining their stimulation capacity) such as a dose in the range of about 3,000 to 7,000 rads, preferably about 5,000 rads. Adherent peripheral blood monocytes are isolated from the cell suspension. These adherent cells are loaded with peptide by mixing the cells with about 10 ng/ml to 10 μg/ml of each peptide.

Next the CD8⁺ cells are combined with the adherent peripheral blood monocytes at a ratio of about ten CD8⁺ cells to one peripheral blood monocyte. In a preferred embodiment, the cells are incubated for about six to seven days. The combined cells can be further incubated with IL-2 and IL-7 in media. Alternatively, it is possible to stimulate the suspension of CD8+ cells in a non-specific manner such as by contacting the cells with a mAb directed against the CD3 receptor in the presence of -irradiated “feeder cells” composed of peripheral blood mononuclear cells (PBMCs) or alternatively, CD8-depleted PBMCs using a dose in the range of about 3,000 to 4,000 rads, preferably about 3,500 rads to irradiate the feeder cells.

The CTL population can be expanded further in the presence of growth factors. In addition, the CD8⁺ suspension can be assayed for suitable CTL activity as well as for CTL purity, sterility and endotoxin content. Once the cells are found to be satisfactory, they can be introduced into a subject. Methods for introducing or reintroducing cells into a subject are known in the art, such as those methods that are used to repopulate bone marrow as part of a bone transplantation procedure or those methods used in the clinical trial using CD34+ cells (see Amado, et al. (1999). Human Gene Therapy, 10:2255-2270). Preferably a dose of cells of between 6-10×10⁹ cells are used per treatment and the treatment may be repeated between 1 and 6 times.

Like other CD8+ T-lymphocytes and CD4+ T-lymphocytes, CTLs are dependent on interleukin-2 for survival and growth. Therefore, in one embodiment, the subjects will have interleukin-2 administered to them prior to or alternatively after the administration of the CTL cell population. Interleukin-2 will likely increase the survival, replication and activity of the CTLs and can also increase survival of other lymphocytic cell types that may be introduced into the subject. In a preferred embodiment, the subject will receive 3 MIU of IL-2 before and/or after administration of the cell population.

High doses of IL-2 appear to be more effective than low dose continuous infusions, yet high doses of IL-2 are also more toxic. The most common side effects are influenza-like symptoms. The most severe side effects are hypotension, capillary leak syndrome, and reduced organ perfusion. IL-2 has been used clinically for both renal cell carcinoma and malignant melanoma. The combination of IFN-α-2b and IL-2 in low dose subcutaneous regimens has been described in other clinical settings (Pectasides et al., Oncology(1998) 55:10-15; Piga et al., Cancer Immumol Immunotherapy (1997) 44:348-351).

In a modified version of the present invention, the CD8+ cells produced according to the methods of the present invention are introduced to a subject in combination with CD4+ T-lymphocytes for the treatment of infectious disease. In this aspect, the CTL population will be produced in the same way as in the first aspect and will be processed concurrently with, though at all times subsequent to separation, separately from, the CD4+ population. The CD4+ population is preferably also transduced with the same infectious disease protective gene therapeutic as that with which the CD8+ cells were transduced.

In yet another version of the present invention, the CD8+ cells produced according to the methods of the present invention are introduced to a subject in combination with CD34+ hematopoietic progenitor cells for the treatment of infectious disease. In this aspect, the CTL population will be produced in the same way as in the first aspect and will be processed concurrently with, though at all times subsequent to separation, separately from, the CD34+ HP cell population. The CD34+ HP population is preferably also transduced with the same infectious disease protective gene therapeutic as that with which the CD8+ cells were transduced.

In another preferred embodiment where the peptides and methods are directed toward the control of viral disease, the administration of the cells is combined with the use of antiretroviral treatment withdrawals (i.e., treatment interruptions). The term “antiviral treatment interruptions” refers to the cessation of non-cell therapy antiviral treatment for a period of time and during the non-cell therapy antiviral treatment interruption the methods of this invention are employed or continued.

It is contemplated that the methods of this invention are useful for infectious diseases of the hematopoietic system generally. However, in a particular application, the invention is directed towards therapy for HIV, wherein the harvested cells from a HIV positive subject are enriched for the various cell types and the therapeutic gene(s) encodes an anti-HIV product(s).

The present invention will now be further described with reference to a number of examples.

### EXAMPLE 1

### The Use of Non-Naturally Occurring Antigen-Presenting Cell (nnAPC) Derived from Drosophila melanogaster Cells for the Generation of HIV-Specific CTLs

The non-naturally occurring antigen-presenting cell (nnAPC) are produced from Drosophila melanogaster cells that were modified to express human class I HLA binding and co-stimulatory molecules according to the methods disclosed in PCT publication PCT/US02/005748. These cells are capable of presenting up to fifteen different peptide molecules, preferably peptide molecules that are simultaneously exogenously loaded onto the surface of the previously transfected Drosophila cells. A Drosophila cell line, which may be used in this way, can be produced as follows:

The Schneider S2 cell line was prepared from Drosophila melanogaster (Oregon-R) eggs according to published procedures and has been deposited with the American Type Culture Collection as CRL 10974. S2 cells are grown in commercial Schneider's Drosophila medium supplemented with 10% fetal bovine serum.

The pRmHa-3 plasmid vector for expressing MHC Class I and co-stimulatory proteins in S2 cells was derived from the pRmHa-1 expression vector as described in the literature. The vector contains a metallothionein promoter, metal response consensus sequences and an alcohol dehydrogenase gene bearing a polyadenylation signal isolated from Drosophila melanogaster. The plasmid vector pRmHa-3 plasmid is modified to include complementary DNA sequences for human class I HLA A2.1, B7.1, B7.2, ICAM-1, β-2 microglobulin and LFA-3, wherein A2.1 can be substituted with any human class I DNA sequence.

Complementary DNAs for Transfection were Prepared as Follows:

HLA-A2.1 and β-2 microglobulin: Reverse transcription-PCR from K562 cells using primers derived from the published sequence
B7.1: Reverse transcription-PCR from K562 cells using primers derived from the published sequence
ICAM-1: Reverse transcription-PCR from K562 cells using primers derived from the published sequence
B7.2: Reverse transcription-PCR from HL-60 cells (ATCC CCL-240) using primers derived from the published sequence
LFA-3: Reverse transcription-PCR from HL-60 cells (ATCC CCL-240) using primers derived from the published sequence

The S2 cells are transfected with a phsneo plasmid and said pRmHa-3 plasmid containing complementary DNA. Stably transfected cells are selected by culturing in Schneider's medium containing geneticin. Twenty-four hours before use expression of the transfected genes was induced by the addition of CuSO₄.

The insect cells of the present invention are grown in a media suitable for growing insect cells, hereinafter termed “insect growth media”. Insect growth media are commercially available from a number of vendors, such as, Schneider™'s Drosophila Medium, Grace's Insect Media, and TC-100 Insect Media. Alternatively, insect growth media can be prepared by one of ordinary skill in the art. Typically the media will include components necessary to promote and sustain the growth of insect cells, such as, inorganic salts (for example, calcium chloride, magnesium sulfate, potassium chloride, potassium phosphate, sodium bicarbonate, sodium chloride, and sodium phosphate), amino acids, various carbohydrate and chemical species (Schneider, Imogene (1964) Exp. Zool. 156:1:91). Alternatively, the media can also include vitamins, minerals, and other components that aid in the growth of insect cells.

This Drosophila cell line is used to make nnAPCs as follows:

The Drosophila cell line is loaded with at least one and up to fifteen different exogenous peptides where each peptide is preferably eight to ten amino acids in length. The peptides are peptide molecules containing immunostimulatory epitopes, preferably epitopes that can be demonstrated in vitro to stimulate CTL activity. The epitopes are preferably identified from infectious agents, preferably viruses and more preferably, an immunodeficiency virus, such as, HIV. The following peptides are examples of what may be used for HIV/AIDS and these have been validated by showing they elicit and in vitro CTL activity: HLA-A2 T cell epitopes for HIV (ILKEPVHGV HIVpol and SLYNTVATL HIVgag). These two epitopes were validated by generating specific CTLs following stimulation with nnAPCs loaded with those two peptides. Other examples of HIV epitopes that may be used are as found in Part IIA “HIV CTL Epitope Tables”; Part IIB “HIV CTL Epitope Maps”; Part IIC “References” of HIV Molecular Immunology 2001 (Eds Korber et al) Division of AIDS, NIAID http://hiv-web.lanl.gov/immunology.

### EXAMPLE 2

### Isolating CD8+ Cells

CD8⁺ cells are isolated from leukapheresis samples by positive selection using the Dynabeads™ isolation procedure (Dynal). An anti-human CD8 mouse monoclonal antibody (50 μg/ml in human gamma globulin [Gammagard®]) is added to washed cells in Dulbecco's PBS supplemented with 1% human serum albumin (Baxter-Hyland) and 0.2% Na citrate. After incubation at 4° C. for forty-five minutes with gentle mixing, the cells are washed and re-suspended in the same buffer containing Dynal magnetic beads (Dynabeads™) coated with sheep anti-mouse IgG at a bead to cell ratio of 1:1. The cells and beads are placed into a sterile tube and gently mixed at 4° C. for forty-five minutes. At the end of this time, the antibody-bound cells are removed magnetically using the MPC-1® separator according to the manufacturer's instructions (Dynal). Dissociation of the CD8 cell-bead complex is achieved by incubation at 37° C. for forty-five minutes in the presence of CD8 peptide₅₉₋₇₀ (AAEGLDTQRFSG; SEQ ID NO: 12). Free beads are removed magnetically and the CD8 cells are counted and analyzed by flow cytometry to evaluate purity.

### EXAMPLE 3

### Purification and Sensitization of CD8+ Cells

Transfected Drosophila S2 cells are incubated in Schneider's medium (10⁶ cells/ml) supplemented with 10% fetal calf serum and CuSO₄ at 27° C. for twenty-four hours. Cells are harvested, washed and re-suspended in Insect X-press medium (BioWhittaker) containing 100 μg/ml human tyrosinase₃₆₉₋₃₇₇. Following incubation at 27° C. for three hours, the S2 cells are mixed with CD8⁺ cells at a ratio of 1:10 in RPMI medium (Gibco) supplemented with 10% autologous serum. The cell mixture is incubated for preferably at least four days and more preferably between 6-7 days at 37° C. during which the Drosophila cells die off. On day five, IL-2 (20 U/ml) and IL-7 (30 U/ml) are added to selectively expand the HIV-specific CTL population.

### EXAMPLE 4

### Transduction of CD8+ T-lymphocytes

In one embodiment, the CTL enriched CD8+ T-lymphocytes are transduced with an infectious disease protective gene therapeutic, for example, a ribozyme conferring protection to HIV, such as RRz2. Such transduction is carried out, for example, by means of a retrovirus, such as, LNL6 to involve the introduction of the genetic agent, such that it is expressed in the cells transduced and in their subsequent progeny cells (e.g., Knop et al (1999).

### EXAMPLE 5

### Restimulation of Cells with PBMCs

Autologous, CD8-depleted PBMCs, obtained at the time of leukapheresis, are thawed (if frozen), washed and re-suspended at 10⁶ cells/ml in RPMI medium containing 10% autologous serum (as a source of β2 microglobulin) and 10-50 μg/ml of stimulatory HIV peptide. Following γ-irradiation (5,000 rads), the cells are incubated at 37° C. for two hours. Non-adherent cells are removed by washing with Dulbecco's PBS. Adherent monocytes are loaded with the peptide by incubation for 90 minutes in Hepes-buffered RPMI medium containing 10% autologous serum and 10 ng/ml-10 μg/ml of one or more HIV-stimulatory peptides such as HIV (ILKEPVHGV HIVpol and SLYNTVATL HIVgag). Other examples of HIV epitopes that may be used are as found in Part IIA “HIV CTL Epitope Tables”; Part IIB “HIV CTL Epitope Maps”; Part IIC “References” of HIV Molecular Immunology 2001 (Eds Korber et al) Division of AIDS, NIAID http://hiv-web.lanl.gov/immunology. The supernatant is removed and the Drosophila-activated CD8⁺ cell suspension (3×10⁶ cells/ml in RPMI medium with 10% autologous serum) is added at a ratio of 10 CD8⁺ cells to 1 adherent monocyte. After three to four days of culture at 37° C., IL-2 (20 U/ml) and IL-7 (30 U/ml) are added with a medium change to selectively expand the HIV-specific CTL population.

Non-Specific Expansion

Optionally effector cells are non-specifically expanded by culturing them in RPMI medium supplemented with autologous serum, anti-CD3 monoclonal antibody (OKT®3), and IL-2 and γ irradiated autologous PBMCs (such as a dose in the range of about 3,000 to 4,000 rads, preferably about 3,500 rads) or alternatively, CD8-depleted PBMCs alone or IL-2 and γ irradiated autologous PBMCs (such as a dose in the range of about 3,000 to 4,000 rads, preferably about 3,500 rads) alone.

Reagents

| REAGENT | SUPPLIER | GRADE | NOTES |
|---|---|---|---|
| Rh IL-2 | Chiron | USP | sterile solution |
| Rh IL-7 | Genzyme | Research | lyophilized, sterile |
| | | | solution |
| Human | | Research | |
| tyrosinase ₃₆₉₋₃₇₇ | | | |
| Dynabeads ® | Dynal | GMP | sheep anti-mouse IgG |
| M-450 | | | magnetic beads |
| Human serum | Baxter | USP | sterile, non-pyrogenic |
| albumin | | | hepatitis virus-free, 25% |
| | | | solution |
| fetal bovine | Gemini | Research | sterile, BSE-, endotoxin-, |
| serum | | | mycoplasma-free |
| Gammagard ® | Baxter | USP | sterile, human immune |
| | | | globulin solution for |
| | | | injection |
| Anti-CD8 | | Research | mouse anti-human CD8 |
| antibody | | | monoclonal antibody |
| CD8 | | Research | release of CD8⁺ cells |
| peptide ₅₉₋₇₀ | | | from magnetic beads |
| W6/32 | ATCC | Research | mouse anti-human HLA-A, |
| | | | B, C monoclonal antibody |

Assays for Activity and Purity
CTL Assay

Malme 3M cells (ATCC) are used as target cells in a ⁵¹Cr release assay. 5×10⁶ Malme 3M cells in RPMI medium containing 4% fetal calf serum, 1% HEPES buffer and 0.25% gentamycin are labeled at 37° C. for one hour with 0.1 mCi ⁵¹Cr. Cells are washed four times and diluted to 10⁵ cells/ml in RPMI with 10% fetal bovine serum (HyClone). In a 96-well microtiter plate, 100 μl effector CTLs and 100 μl peptide-loaded, ⁵¹Cr-labeled Malme 3M target cells are combined at ratios of 100:1, 20:1 and 4:1 (effector:target). K562 cells are added at a ratio of 20:1 (K562:Malme 3M) to reduce natural killer cell background lysis. Non-specific lysis is assessed using the non-tumor HLA-A2.1 fibroblast cell line, Malme 3. Controls to measure spontaneous release and maximum release of ⁵¹Cr are included in duplicate. After incubation at 37° C. for six hours, the plates are centrifuged and the supernatants counted to measure ⁵¹Cr release.

Percent specific lysis is calculated using the following equation:

$$\frac{cpm sample - cpm spontaneous release}{cpm maximum release - cpm spontaneous release} \times 100$$

Flow Cytometry.

CD8⁺ cells, before and after in vitro activation are analyzed for a number of cell surface markers using fluorescent monoclonal antibodies and FACS analysis.

### EXAMPLE 6

### Preparation of CD4+ Cells

Infectious agent-specific CD8+ derived CTLs can also be used in combination with CD4+ T-lymphocytes for the treatment of infectious disease. In this aspect, the CTL population is produced as described above and is processed concurrently with, though at all times subsequent to separation, separately from, the CD4+ population. The CD4+ population is prepared and used as follows. The CD4+ cells are harvested from a subject, subsequent to the isolation of CD8+ cells from the apheresis product. CD4+ cells are transduced with retrovirus, activated and introduced into tissue culture. CD4+ cells are stimulated with materials such as OKT3 and IL-2. The cells are transduced with the anti-infectious disease therapeutic at day 3-5 of culture. The CD4+ cells can also be further stimulated and expanded using IL-2 and appropriate medium changes. The CD4+ cells are harvested, washed, checked for sterility and infused into the patient.

An example of this technique is as follows: approximately 1×10⁸ mononuclear cells/kg were leukapheresed from HIV patients using a COBE Spectra (Gambro BCT, Sweden) and CD4+ T-lymphocytes were isolated by Ficoll/Hypaque (Amersham Pharmacia Biotech, Sweden) density gradient medium. The leukapheresed cells were then CD8⁺ T-cell depleted using CD8 CELLector flasks (RPR Gencell, USA). Pre- and post-depletion samples were stained with CD4-FITC, CD8-PE and CD3-perCP or isotype control antibodies (BDIS, USA) for T-cell subset analysis by flow cytometry (Knop et al, 1999).

The CD4⁺ enriched T-lymphocytes were then incubated in 15 ml of either control (LNL6) or ribozyme (RRz2) GMP-grade virus (in duplicate) for 15-30 minutes at 37° C. and stimulated with OKT3 (anti-CD3) antibody (Orthoclone; Janssen-Cilag, Belgium) at a concentration of 50 ng per 10⁷ cells.

The enriched CD4⁺ T-lymphocytes were then introduced into four pre-cultured Peripheral Blood Lymphocyte-MPS artificial capillary cartridges (Cellco Cellmax™, USA) by syringing the 15 ml cell suspension into a cartridge side port. The cells were cultured initially in AIM-V medium (Gibco BRL) containing 5% heat inactivated autologous plasma. At day 2 of culture, 100 U/ml IL-2 (rIL-2; Chiron, USA) was added to the culture medium and maintained until harvest. The proportion of plasma was reduced stepwise by using non-plasma containing media for subsequent media additions, and cultures were almost plasma free by day 7 (less than 0.1% remaining). By day 4 of culture, media additions to the reservoirs were AIM-V only. To monitor daily cell growth and the status of the medium, the concentration of L-lactate in the module reservoir was determined by taking a sample from the reservoir and measuring the L-lactate concentration (L-lactate reagent Sigma, USA). This determination was based on the approximation that 4×10⁶ PBL produces 1 mg lactate per day (Cellco). The medium in the reservoir was changed (in order to add fresh growth additives and remove metabolites) when the concentration of lactate exceeded 0.6 mg/ml.

The second retroviral transduction was conducted when the target CD4⁺ lymphocytes reached exponential growth phase (generally days 3-5 of culture) as monitored by lactate concentration. This was conducted as two separate transductions performed on consecutive days (30 ml VCM was added in the first transduction and 70 ml VCM in the second). Transduction was effected by direct injection of VCM through the cartridge sideports as previously described (Knop et al, 1999). Prior to injection into the sideport, 100 U/ml IL-2 and protamine sulphate (Fisons/Rhone-Poulenc Rorer, USA) were added to the VCM and protamine sulphate also added to the culture reservoir. The concentration of protamine sulphate added yielded a final concentration of 5 μg/ml.

On day 8 of culture, two days prior to infusion, samples were removed from each of the cartridges and reservoirs for pre-harvest testing. The analyses conducted were mycoplasma PCR ELISA (Boehringer Mannheim/Roche, Switzerland), flow cytometry analysis (CD4, CD8 and CD3) and sterility testing (Sydpath, St Vincent's Hospital pathology services, Australia). Samples were also removed for quantitative competitive PCR determination of percentage marked cells (as an indication of transduction) as previously described (Knop et al, 1999).

Cells were harvested when cultures had reached plateau growth phase (generally day 10 of culture), as determined by no further increase in L-lactate production over two consecutive days. The cells were harvested, according to the Manufacturer's instructions, by injecting media into the extra-capillary space through the capillary pores and flushing the cartridges with medium. This was followed by three cell washes (centrifugation at 200 g for 5-minutes) then resuspension in an infusion buffer containing 0.18% saline, 4% glucose (Baxter) and 2.5% human serum albumin (Albumex 20, Red Cross Blood Bank, Australia). Samples were removed at this time for further testing: sterility (aerobic and anaerobic culture), analysis by flow cytometry and archival storage. Samples of the final cell infusate were sent to BioReliance Corporation (formerly Magenta, Rockville, USA) for biological replication-competent retrovirus (RCR) testing, using the PG4 S+L− focus assay following amplification in the Mus dunni cell line. For each patient, the four cell populations (2 of LNL6 and 2 of RRz2 infected cells) were finally pooled, resuspended at approximately 2×10⁹ cells in a total volume of 100 ml and injected into a transfusion bag containing 1000 ml of the infusion buffer. The cell product was infused within 2 hours of harvest into the matching HIV-1 positive twin over a 1 hour period.

### EXAMPLE 7

### Preparation of CD34+ Cells

The present invention also provides for the use of infectious agent-specific CD8+ derived CTLs in combination with CD34+ HP cells (also termed HP cells) for the treatment of infectious disease. In this aspect, the CTL population will be produced in the same way as in the first aspect and will be processed concurrently with, (though at all times subsequent to separation) separately from the CD34+ population. The CD34+ population can be prepared and used for example by obtaining from said subject a population of viable cells comprising hematopoietic progenitor (HP) cells of the CD34+ class, treating and/or culturing said population of cells to provide a pool of cells comprising a portion of HP cells in excess of 40%, and introducing said therapeutic gene(s) into at least a portion of the HP cells within said pool of cells such that said therapeutic gene(s) is/are capable of being expressed in said portion of HP cells wherein a resultant pool of viable cells is prepared which includes sufficient HP cells including the therapeutic gene(s) such that, upon delivery to said subject, the subject receives a dose of at least 0.5×10⁶ CD34+ HP cells containing the therapeutic gene(s) per kg body weight.

Preferably, the resultant pool of viable cells are transduced with a disease-treating gene therapeutic and the viable cell pool comprises sufficient therapeutic gene(s) containing CD34+ HP cells such that, upon delivery to said subject, the subject receives a dose of at least 5×10⁶, more preferably in excess of 2×10⁷, and even more preferably in excess of 5×10⁷ therapeutic gene(s) containing CD34+ HP cells/kg body weight.

Preferably, the resultant pool of viable cells is such that, upon delivery to said subject, the subject receives a total number of cells (i.e. the HP cells containing the therapeutic gene(s) and all other cells present in the resultant pool of cells) of at least 1×10⁷/kg body weight up to 4×10⁷ cells/kg or more preferably up to 1×10⁷ kg or more).

The population of cells “harvested” from the subject may be obtained by any of the methods well known in the art. For instance, the subject may be treated so as to mobilize HP cells from bone marrow into the peripheral blood (e.g. by administering a suitable amount of the cytokine granulocyte—colony stimulating factor, G-CSF) followed by apheresis filtration. Alternatively, HP cells may be aspirated from bone marrow or cord blood in accordance with well-known techniques.

Treatment of the harvested population of cells may include one or more washing steps (e.g. using centrifugation or automated cell washers) and/or de-bulking steps (i.e. to remove excess red blood cells, granulocytes, platelets, T-lymphocytes and), by use of a device such as the Dendreon DACS System (Charter Medical, Winston Salem, N.C.) and, preferably, a HP cell selection step. HP cell selection may be achieved by immune affinity or flow cytometry techniques. Preferably, the HP cell selection step selects CD34+ cells or in another embodiment may involve antigen depletion of mature/committed hematopoietic cells, thereby enriching for HP cells. Such cells can be selected using a variety of selection devices such as, but not limited to, the Nexell/Baxter Isolex 300I (Irvine, Calif.), the Miltenyi CliniMACS,(Miltenyi; Biotech GmBH, Bergisch Gladbach, Germany), Stem Cell Technologies (Vancouver, BC, Canada) StemSep Device.

The treatment of the harvested population of cells may also involve a cell culturing step to increase cell numbers and especially to increase the number of selected HP cells. Cell culturing is also required to introduce the therapeutic gene(s) and cell culturing may additionally be conducted after introduction of the therapeutic gene(s) into at least a portion of the HP cells to expand the number of such gene(s)-containing HP cells.

The initial treatment steps (mobilization, apheresis, HP selection) results in the obtaining of, and enriching for, HP cells. The definition of the percentage of HP cells requires a measurable aspect of these cells such as CD34 antigen positivity. It is to be understood that the treated pool of cells comprises at least 40%, more preferably at least 60% and most preferably at least 80%, HP cells.

Introduction of the therapeutic gene(s) or nucleic acid sequence(s) into at least a portion of the HP cells may be achieved with any of the methods well known to the art, but most conveniently through transduction using retroviral vectors or other viral or non-viral (DNA or RNA) vectors carrying the therapeutic gene(s) or nucleic acid sequence(s), and, preferably, a transduction-facilitating agent (e.g. for retroviral vectors, the CH296 fragment of fibronectin known as RetroNectin). The HP cells containing the therapeutic gene(s) or nucleic acid sequence(s), and cells derived therefrom (i.e. from subsequent lymphoid and myeloid hematopoiesis), contain and are capable of expressing the therapeutic gene(s) or nucleic acid sequence(s).

In a preferred embodiment infusion is performed at day 3 post introduction to cell culture.

For each of these aspects, in the case of HIV/AIDS, the therapeutic gene(s) may encode a product selected from proteins (e.g. transdominant proteins and intracellular antibodies), antisense molecules (eg antisense RNA), RNA decoys, aptamers, interfering RNA and catalytic ribozymes.

A preferred method for the isolation, culture, transduction and re-infusion of CD34+ hematopoietic cells is provided below.

The first step of this procedure uses an agent to mobilize HP cells from the bone marrow into the peripheral blood. An example here is the use of Granulocyte Colony Stimulating Factor (G-CSF, Neupogen™), which is administered to the patient subcutaneously, at least at 10 μg/kg/day and preferably at 30 μg/kg/day, once daily, for up to five consecutive days. Complete Blood Counts (CBCs), differential and platelet count are performed daily during G-CSF administration to assess the extent of the leucocytosis. A blood sample for CD34+ cell count is drawn on day 3 of G-CSF administration to ensure that the peripheral blood CD34+ count is greater than 20 cells/mm³ prior to the start of apheresis. Failure to attain this CD34+ cell number does not however prevent apheresis on days 5 and 6 of G-CSF administration.

Apheresis is a method of “blood filtration” to obtain the mononuclear cell fraction of the peripheral blood. It is conducted with a Cobe Spectra (Gambra), Hemonetics (Domedica) or Amicus (Baxter) machines on at least two separate occasions, (preferably on days 5 & 6 following mobilization, where day 1 is the first day of induced mobilization), though in other examples this can be done on earlier or later days by determining the day at which the peripheral blood CD34+ count is greater than 5 cells/mm³ or more preferably 10 cells/mm³ and most preferably 20 cells/mm³. In a preferred embodiment, this apheresis yields cellular product from about 5 Liters (L) of blood flow, though preferably this will be 5-10 L, but more preferably 10-20 L, and more preferably still 20 L or greater. Product from each apheresis is either treated separately or, in a preferred embodiment, pooled after the second apheresis. Total cell counts, and absolute CD34+ cell numbers are recorded. Use of these aphereses will produce up to greater than 5×10⁶, preferably greater than 2×10⁷, more preferably greater than 4×10⁷ HP (as measured by CD34 positivity) cells/kg

The pooled cells are washed. This is done by cell centrifugation or more preferably using an automated cell washer, in one example this cell washing is done by using a Nexell CytoMate washer.

In one embodiment, the cells from the apheresis procedure(s) are “de-bulked”-using a system like a Charter Medical DACS-SC™ system. In the embodiment where product is stored overnight from the first day for pooling with second day product, the two apheresis products are de-bulked on the day of collection and the first product stored until the second product has been de-bulked.

The cells are taken, pooled (in the embodiment where there are two products) and washed by centrifugation or by using a Nexell CytoMate device or similar. (If there are more than two products all will be pooled at the latest time point).

CD34+ cells are selected from the post-washing product by using the Isolex 300i, Miltenyi or a lineage depletion strategy of cells expressing markers (e.g. CD2, CD3, CD14, CD16, CD19, CD24, CD56, CD66b glycoprotein A, StemSep). The enriched pool of CD34+ or lineage depleted cells preferably comprises at least 40%, more preferably at least 60% and most preferably at least 80% cells of this type.

The cells are washed by centrifugation or by using the Nexell CytoMate or similar.

The cells are counted and placed at preferably 1×10⁵ to 5×10⁶ cells/ml into cell culture flasks, cell culture bags or in a preferred embodiment into 1,000 ml (390 cm²) Nexell Lifecell X-Fold Culture Bag or similar with Iscove's Modified Dulbecco's Medium plus 10% Fetal Bovine Serum (FBS) containing cytokines/growth factors. In a preferred embodiment this cytokine/growth factor mixture consists of Stem Cell Factor (50 ng/ml) and Megakaryocyte Growth and Development Factor 100 ng/ml). Steps 3-9 will result in up to 12×10⁷ HP cells or more (as assessed by CD34 positivity) per kg.

The cells are harvested from the first flask, tissue culture bag, including a preferred embodiment of a Lifecell Culture Bag or similar and using the Cytomate device or similar, resuspended in retroviral supernatant (an example of this is a 200 ml aliquot) and transferred into a second tissue culture container, one type of which is the Lifecell X-Fold Culture Bag which have a retrovirus transduction facilitating agent. Such agents include polybrene, protamine sulphate, cationic lipids or in a preferred embodiment, in a tissue culture container that has been pre-coated with RetroNectin at 1-4 mcg/cm². After 4-10 hours or up to 24 hours, the transfer procedure will be repeated using the CytoMate or similar; for this second transduction cells are either transferred to a new tissue culture container (polybrene, protamine sulphate) or returned to the same or similar RetroNectin—coated container from which they came. In a preferred embodiment, this is done in a fresh aliquot of retroviral supernatant and cultured overnight. In other embodiments, this is either not done or repeated several times for similar periods of time. An aliquot of the retroviral supernatant(s) is collected for sterility testing. This will result in up to 6×10⁷ gene-containing HP cells or more (as assessed by CD34 positivity) per kg. This number is determined by quantitative assay such as DzyNA PCR. The transduction efficiency will be at least 20%, and preferably in the range from 30-50%, and more preferably greater than 50%.

On the morning of day 9, cells are harvested and washed using standard cell centrifuge or automated systems such as the Cytomate samples of cell culture. This will yield up to 5.7×10⁷ gene-containing HP cells or more (as assessed by CD34 positivity) per kg.

Cells are resuspended in a physiologic infusion buffer containing 5% human serum albumin or similar as carrier. Aliquot samples are removed for sterility (aerobic, anaerobic, fungal, mycoplasma). Infusion product is not released until the results of endotoxin (LAL) and gram stain testing are available.

The CD34+ cell preparation is administered to the patient pre-medicated as appropriate. In a preferred embodiment, the patient receives a single infusion of 0.5-6×10⁷ transduced CD34+ cells per kilogram of body weight (cells/kg) in the physiologic infusion buffer containing 5% human serum albumin or similar as carrier. The dose of transduced CD34+ cells per patient will depend on the efficiency of each step of the mobilization, apheresis, isolation, culture and transduction procedures. The total number of CD34+ cells (transduced and non-transduced) is determined by cell counting and flow cytometry. The introduced gene-containing HP cells give rise to a chimeric hematopoietic system in which there is a percentage of gene-containing HP cells in the bone marrow. In a preferred embodiment, the one for the treatment of HIV/AIDS, this percentage of gene-containing HP cells is at least 5%, preferably greater than 10% and more preferably greater than 20%.

In a preferred embodiment, the subject does not require myeloablation of the bone marrow or other marrow conditioning regimen, and the step of delivering the cells results in the subject receiving a dose of at least 0.5×10⁶ CD34+ cells containing the therapeutic gene(s)/kg body weight. In the Phase I CD34+ clinical trial it was found that this is a “sufficient” dose of cells to produce a chimeric hematopoietic system that will yield persistence (presence of gene-containing cells for greater than one year post-infusion) of anti-HIV product-containing mature lymphoid (CD4+ and CD8+ T-lymphocytes) and myeloid (monocyte/macrophages) cells.

### EXAMPLE 8

### Use of IL-2

IL-2 is used post infusion to benefit treatment. A minimum dose of IL-2 contemplated for use is 2-4 MIU/m2 (million international units per meter squared), subcutaneously for 12 days every 3 weeks and preferably 3 MIU/m². Immune activation is monitored and significant increases in lymphocytes, activated CD4+ and CD8+ T cells, NK cells, and monocyte DR expression are monitored.

The presence of the high affinity IL-2 receptor (CD25/CD122/CD132) on the surface of the CD8+ cells suggests that these cells are capable of responding to low doses of IL-2 in vivo.

### EXAMPLE 9

### Infusion of Cells

The cells are delivered to the subject in accordance with routine methods such as cell infusion. The cells are preferably delivered with a pharmacologically-acceptable carrier (e.g. 5% Human Serum Albumin). The subject may or may not be first (ie before re-infusion of the cells) subjected to myeloablation of the bone marrow or other hematopoietic conditioning regimens.

### EXAMPLE 10

### Monitoring Expession of the Gene Construct

The presence of the gene construct transduced into infused cells can be monitored via a number of methods. In a preferred method quantitative real time PCR methodology (DzyNA-PCR) is used. DzyNA-PCR (Todd et al. 2000) and U.S. Pat. Nos. 6,140,055 and 6,201,113 provide a general strategy for the detection of specific genetic sequences associated with disease or the presence of foreign agents. The method provides a system that allows homogeneous nucleic acid amplification coupled with real-time fluorescent detection in a single closed vessel. The strategy involves in vitro amplification of genetic sequences using a DzyNA primer which harbors the complementary (antisense) sequence of a 10:23 DNAzyme (Santoro et al. 1997). During amplification, amplicons are produced which contain active (sense) copies of DNAzymes that cleave a reporter substrate included in the reaction mix. The accumulation of amplicons during PCR is monitored by changes in fluorescence produced by separation of fluoro/quencher dye molecules incorporated into opposite sides of a DNAzyme cleavage site within the reporter substrate. Cleavage of this reporter substrate indicates successful amplification of the target nucleic acid sequence. Real-time measurements can be performed on the ABI PRISM® 7700 Sequence Detection System (Applied Biosystems) or other thermocyclers that have the capacity to monitor fluorescence in real time.

DzyNA-PCR is a general strategy for the detection of specific genetic sequences associated with disease or the presence of foreign agents. The method provides a system that allows homogeneous nucleic acid amplification coupled with real time fluorescent detection in a single closed vessel. The strategy involves in vitro amplification of genetic sequences using a DzyNA primer which harbors the complementary (antisense) sequence of a 10:23 DNAzyme. During amplification, amplicons are produced which contain active (sense) copies of DNAzymes that cleave a reporter substrate included in the reaction mix. The accumulation of amplicons during PCR is monitored by changes in fluorescence produced by separation of fluoro/quencher dye molecules incorporated into opposite sides of a DNAzyme cleavage site within the reporter substrate. Cleavage of this reporter substrate indicates successful amplification of the target nucleic acid sequence. Real time measurements can be performed on the ABI Prism 7700 Sequence Detection System or other thermocyclers that have the capacity to monitor fluorescence in real time (eg Corbett Rotor-Gene, Stratagene Mx 4000, Cepheid SmartCycler, Roche LightCycler, Biorad iCycler etc).

DzyNA PCR protocols have been developed for analysis of vectors and therapeutic agents that contain the neomycin resistance gene. This assay has various uses including estimation of the percent transduction of cells and monitoring the presence and quantification of transduced cells, or their progeny, within patients undergoing gene therapy.

The reporter substrate, Sub G5-FD, was synthesised by Trilink Biotechnologies (California, USA). Sub G5-FD (illustrated below) is a chimeric molecule containing both RNA (shown below in lower case) and DNA nucleotides. It has a 3′ phosphate group that prevents its extension by DNA polymerase during PCR. Sub G5-FD was synthesised with FAM (F) and DABCYL (D) moieties attached to the “T” deoxyribonucleotides indicated. The cleavage of the reporter substrate can be monitored at 530 nm (FAM emission wavelength) with excitation at 485 nm (FAM excitation wavelength).

SubG5-FD is shown here:

5′CACCAAAAGAGAAC(T-F)GCAATguT(T-D)
CAGGACCCACAGGAGCG-p 3′

Two PCR primers were synthesised by Sigma Genosys (NSW, Australia). The 5′ PCR primer (5L1A) hybridizes to the neomycin resistance gene. The 3′ primer (3L1Dz5) is a DzyNA PCR primer, which contains (a) a 5′ region containing the catalytically inactive antisense sequence of an active DNAzyme and (b) a 3′ region, which is complementary to the neomycin resistance gene. During PCR amplification using 5L1A and 3L1Dz5, the amplicons produced by extension of 5L1A contain both neomycin resistance sequences and catalytically active sense copies of a DNAzyme incorporated in their 3′ regions. The active DNAzyme is designed to cleave the RNA/DNA reporter substrate Sub G5-FD.

The sequences of the PCR primers is shown here:

5L1A (5′ primer)
5′ GAG TTC TAC CGG CAG TGC AAA 3′
3L1Dz5 (3′ DzyNA primer)
5′ CAC CAA AAG AGA ACT GCA ATT CGT TGT AGC TAG CCT
TTC AGG ACC CAC AGG AGC GGC AAG CAA TTC GTT CTG
TAT C 3′

The human cell line CEMT4 was obtained from the American Type Culture Collection (Rockville, Md.). CEMT4 cells were transduced with retrovirus containing the neomycin resistance gene. Genomic DNA was isolated from CEM T4 cells, as well as CEMT4 cells transduced with retrovirus harboring the Neomycin resistance gene, using the QIAGEN DNeasy Tissue Kit (QIAGEN Pty Ltd, Victoria, Australia. Cat #69504). DNA extracted from transduced cells was mixed with DNA from untransduced cells (by weight) to obtain the following percentage of transduced DNA—100%, 11%, 1.2%, 0.1%, 0.02% and 0% (ie 100% untransduced CEMT4).

Genomic DNA isolated from CEM T4 cells, as well as CEMT4 cells transduced with retrovirus harboring the Neomycin resistance gene, was amplified by DzyNA PCR. Reactions contained 30 pmole 5L1A, 1 pmole 3L1Dz5, 10 pmol Sub G5-FD, 20 U RNasin (Promega, Catalogue # N2515), 20 pmol ROX passive reference dye and 1× QIAGEN HotStarTaq Master mix (QIAGEN Pty Ltd, Victoria, Australia. Catalogue #203445) plus an additional 2.5 mM MgCl₂ in a total reaction volume of 40 μl. Duplicate reactions were set up which contained 1 μg of genomic DNA. Control reactions contained all reaction components with the exception of genomic DNA. The reactions were placed in an ABI Prism 7700 Sequence Detection System, denatured at 95° C. for 10 minutes, subjected to 10 cycles of 70° C. for 1 minute with a temperature decrease of 1° C. per cycle, and 94° C. for 1 minute. This was followed by a further 60 cycles at 60° C. for 1 minute and 94° C. for 30 seconds. Fluorescence was measured by the ABI Prism 7700 Sequence Detection System during the annealing/extension phase of the PCR.

Reactions with genomic DNA containing neomycin resistance gene showed an increase in FAM fluorescence at 530 nm over the fluorescence observed in control reactions. When 1 μg of genomic DNA containing DNA from transduced CEMT4 cells was analysed the calibration curve was linear over the range of 100 to 0.02% transduced cells (R² consistently >0.99). Reactions containing DNA from untransduced cells, or lacking DNA, did increase over the threshold level during 70 thermocycles of PCR. Calibration curves generated using standard amounts can be used to estimate the proportion of cells or DNA, containing the Neomydn resistance gene, in an unknown sample. The experiments described in this example illustrate one set of reaction conditions that can be used to detect and quantify the Neomycin resistance transgene. This protocol can be modified readily by those of ordinary skill in the art and used to detect the RNA transcript from the Neomycin resistance gene following modification of the protocol and inclusion of reverse transcripts in the reaction mix.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

Aguila, H. L., K. Akashi, J. Domen, K. L. Gandy, E. Lagasse, R. E. Mebius, S. J. Morrison, J. Shizuru, S. Strober, N. Uchida, et al. (1997). “From stem cells to lymphocytes: biology and transplantation.” Immunol Rev 157: 13-40.
Cooper, D., R. Penny, G. Symonds, A. Carr, W. Gerlach, L. Q. Sun and J. Ely (1999). “A marker study of therapeutically transduced CD4+ peripheral blood lymphocytes in HIV discordant identical twins.” Hum Gene Ther 10(8): 1401-1421.
Haase, A T, et al. (1996) Quantitative image analysis of HIV-1 infection in lymphoid tissue. Science 274:985-989.

Heslop H E, Ng C Y, Li C, et al. (1996) Long-term restoration of immunity against Epstein-Barr virus infection by adoptive transfer of gene-modified virus-specific T lymphocytes. Nat Med 1996, 2:551-555.
Janewy, C A, Travers, P, Walport, M and Capra, J D (1999) Immunobiology, The Immune System in Health and Disease, 4^{th} Edition, Elsevier
Knop, A. E., A. J. Arndt, M. Raponi, M. P. Boyd, J. A. Ely and G. Symonds (1999). “Artificial capillary culture: Expansion and retroviral transduction of CD4+ T lymphocytes for clinical application.” Gene Ther 6: 373-384.
Kohn, D. B., G. Bauer, C. R. Rice, J. C. Rothschild, D. A. Carbonaro, P. Valdez, Q. Hao, C. Zhou, I. Bahner, K. Kearns, et al. (1999). “A clinical trial of retroviral-mediated transfer of a rev-responsive element decoy gene into CD34(+) cells from the bone marrow of human immunodeficiency virus-1-infected children.” Blood 94(1): 368-371.
Latouche, J. B. and M. Sadelain, M (2000) “Induction of Human Cytotoxic T Lymphocytes by Artificial Antigen-Presenting Cells”, Nature Biotech 18:405-409).
Levinsky, R. J. (1989). “Recent advances in bone marrow transplantation.” Clin Immunol Immunopathol 50(1 Pt 2): S124-132.
Levy, J A (1994) HIV and the Pathogenesis of AIDS, Am Soc Micro, Washington
Marasco, W. A., S. Chen, J. H. Richardson, U. Ramstedt and S. D. Jones (1998). “Intracellular antibodies against HIV-1 envelope protein for AIDS gene therapy.” Hum Gene Ther 9(11): 1627-1642.
McFarland, R. D., D. C. Douek, R. A. Koup and L. J. Picker (2000). “Identification of a human recent thymic emigrant phenotype.” Proc Natl Acad Sci U S A 97(8): 4215-4220.
Murray, J M, Kaufmann, G, Kelleher, A D, Cooper, D A. A model of primary HIV-1 infection. Mathematical Biosciences, 154:57-85 (1998).
Rossi, J. J., E. M. Cantin, N. Sarver and P. F. Chang (1991). “The potential use of catalytic RNAs in therapy of HIV infection and other diseases.” Pharmacol Ther 50(2): 245-254.
Santoro, S. W. and G. F. Joyce (1997). “A general purpose RNA-cleaving DNA enzyme.” Proc Natl Acad Sci USA 94(9): 4262-4266.
Sarver, N., E. M. Cantin, P. S. Chang, J. A. Zaia, P. A. Ladne, D. A. Stephens and J. J. Rossi (1990). “Ribozymes as potential anti-HIV-1 therapeutic agents.” Science 247: 1222-1225.
Schindhelm, K and Nordon, R (1999) eds Ex Vivo Cell Therapy, Academic Press, San Diego
Schwartzberg, L. S., R. Birch, B. Hazelton, K. W. Tauer, P. Lee, Jr., R. Altemose, C. George, R. Blanco, F. Wittlin, J. Cohen, et al. (1992). “Peripheral blood stem cell mobilization by chemotherapy with and without recombinant human granulocyte colony-stimulating factor.” J Hematother 1(4): 317-327.
Sczakiel, G. and M. Pawlita (1991). “Inhibition of human immunodeficiency virus type 1 replication in human T cells stably expressing antisense RNA.” J Virol 65(1): 468-472.
Smythe, J. A., D. Sun, M. Thomson, P. D. Markham, M. S. Reitz, R. C. Gallo and J. Lisziewicz (1994). “A Rev-inducible mutant gag gene stably transferred into T lymphocytes: An approach to gene therapy against human immunodeficiency virus type 1 infection.” Proc Natl Acad Sci USA 91(9): 3657-3661.
Sullenger, B. A., H. F. Gallardo, G. E. Ungers and E. Gilboa (1990). “Overexpression of TAR sequences renders cells resistant to human immunodeficiency virus replication.” Cell 63(3): 601-608.
Sun, L. Q., W. L. Gerlach and G. Symonds (1996). The use of ribozymes to inhibit HIV replication. Catalytic RNA. F. Eckstein and D. Lilley. 10: 329-342.
Sun, L. Q., W. L. Gerlach and G. Symonds (1998). The design, production and validation of an anti-HIV type1 ribozyme. Therapeutic Application of Ribozymes. K. J. Scanlon. Totowa, N.J., Humana Press Inc. 11: 51-64.
Sun, L. Q., J. Pyati, J. Smythe, L. Wang, J. Macpherson, W. Gerlach and G. Symonds (1995a). “Resistance to human immunodeficiency virus type 1 infection conferred by transduction of human peripheral blood lymphocytes with ribozyme, antisense or polymeric trans-activation response element constructs.” Proc Natl Acad Sci USA 92(16): 7272-7276.
Todd, A. V., C. J. Fuery, H. L. Impey, T. L. Applegate and M. A. Haughton (2000). “DzyNA-PCR: use of DNAzymes to detect and quantify nucleic acid sequences in a real-time fluorescent format [see comments].” Clin Chem 46(5): 625-630.
Tough, D. F. and J. Sprent (1995). “Life span of naive and memory T cells.” Stem Cells (Dayt) 13(3): 242-249.
Walter, E A Greenberg P D, Gilbert M J, et al. (1995) Reconstitution of cellular immunity against cytomegalovirus in recipients of allogeneic bone marrow by transfer of T-cell clones from the donor. N Engl J Med 333:1038-1044.
Wang, L., C. Witherington, A. King, W. L. Gerlach, A. Carr, R. Penny, D. Cooper, G. Symonds and L. Q. Sun (1998). “Preclinical characterization of an anti-tat ribozyme for therapeutic application.” Hum Gene Ther 9(9): 1283-1291.

What is claimed is:

1-65. (canceled)

66. A method for producing a cytotoxic T-lymphocyte population primed for virus-specific CTL activity comprising the steps of:
(a) preparing non-naturally occurring antigen-presenting cells (nnAPC) which present at least one virus-specific antigen;
(b) harvesting a population of white blood cells from a subject;
(c) incubating a population of CD8+ cells obtained from the white blood cells in step (b) with the nnAPC cells; and
(d) treating the CD8+ cells with one or more supportive cytokines.

67. The method of claim 66 wherein the nnAPC cells present a plurality of the virus-specific antigens, and have been prepared by incubating the cells with at least two different peptides each comprising one of the virus-specific antigens, respectively.

68. The method of claim 66 further comprising incubating CD8+ cells from step (d) with non-proliferating peripheral blood mononuclear cell-derived adherent cells wherein the adherent cells present one or more of the same virus-specific antigens of step (a).

69. The method of claim 66 further comprising introducing at least one virus-inhibiting nucleic acid into the CD8+ cells.

70. The method of claim 69 wherein the virus-inhibiting nucleic acid is selected from the group consisting of transdominant proteins, intracellular antibodies, antisense molecules, RNA decoys, interfering RNAs, aptamers and ribozymes.

71. The method of claim 70 wherein the virus-inhibiting nucleic acid is a ribozyme.

72. The method of claim 69 wherein the virus-inhibiting nucleic acid is specific for a disease selected from the group consisting of Human papilloma virus, Cytomegalovirus, Epstein Barr Virus, Hepatitis A, Hepatitis B, Hepatitis C, Hepatitis D, Hepatitis E, Measles, Mumps, Polio, Rubella, Influenza, Yellow Fever, Japanese Encephalitis, Dengue, Rabies, Rotavirus, Varicella Zoster, Chikungunya Rift Valley Fever, Respiratory Syncitial Virus, Herpes Simplex, Coronaviruses, Marburg, Ebola, California Encephalitis Virus, JC Virus, Lymphocytic Choriomeningitis Virus, Parvovirus, Rhinovirus, Smallpox, HTLV-1, HTLV-2, and HIV.

73. The method of claim 72 wherein the virus-inhibiting nucleic acid is specific for HIV.

74. The method of claim 69 wherein the virus-inhibiting nucleic acid is passed to CTL progeny.

75. The method of claim 68 wherein the adherent cells are adherent monocytes obtained during the harvesting step (b).

76. The method of claim 75 wherein the adherent monocytes are isolated from a suspension of peripheral blood monocytes after irradiating the suspension with a sufficient dose of γ-radiation necessary to prevent proliferation of the peripheral blood monocytes.

77. The method of claim 68 wherein the incubating step (c) comprises combining the CD8⁺ cells with the adherent peripheral blood monocytes at a ratio of about ten CD8⁺ cells to one adherent peripheral blood monocyte.

78. The method of claim 66 wherein the CD8⁺ cells are tested for at least one parameter selected from the group consisting of cytotoxic T cell activity, CTL cell purity, sterility and endotoxin content.

79. The method of claim 66 wherein the supportive cytokines are selected from the group consisting of IL-2, IL-4, IL-7, IL-15 and IL-21.

80. The method of claim 66 wherein the supportive cytokines are added to the CD8+ cells in step (d) about 4 days or more after step (c) is initiated.

81. A method of claim 66 wherein the nnAPC cells comprise an nnAPC cell line.

82. A method for producing a cytotoxic T-lymphocyte population transduced with virus-inhibiting nucleic acid and primed for virus-specific CTL activity comprising the steps of:
(a) preparing a non-naturally occurring antigen presenting cell line (nnAPC) which presents at least one virus specific antigen;
(b) harvesting CD8⁺ cells from a subject;
(c) incubating the CD8⁺ cells with the nnAPC cell line;
(d) adding Interleukin-2 (IL-2) and Interleukin-7 (IL-7) to the CD8⁺ cells after step (c);
(e) introducing at least one virus-inhibiting nucleic acid into the CD8⁺ cells wherein the virus inhibiting nucleic acid is expressed; and
(f) incubating the CD8⁺ cells with non-proliferating peripheral blood mononuclear cell-derived adherent cells and wherein the adherent cells present at least one of the same virus-specific antigens of step (a).

83. The method of claim 82 wherein the nnAPC cell line presents a plurality of virus-specific antigens, and have been prepared by incubating the cell line with at least two different peptides at least 8 amino acids in length, each peptide comprising one of the virus-specific antigens, respectively.

84. The method of claim 82 wherein the virus-inhibiting nucleic acid is a ribozyme.

85. The method of claim 82 wherein the virus-inhibiting nucleic acid is specific for HIV.

86. The method of claim 82 wherein the virus-inhibiting nucleic acid is passed to CTL progeny.

87. The method of claim 82 wherein the adherent cells are adherent monocytes obtained during the harvesting step (b).

88. The method of claim 87 wherein the adherent cells presenting at least one of the same virus-specific antigens of step (a) are produced by incubating the adherent cells with one or more different peptides, the or each peptide comprising one of the virus-specific antigens, respectively.

89. The method of claim 87 wherein the adherent monocytes are isolated from a suspension of peripheral blood monocytes after irradiating the suspension with a sufficient dose of γ-radiation necessary to prevent further cell proliferation of the peripheral blood monocytes.

90. The method of claim 89 wherein the dose of γ-radiation is in the range of about 3,000 to 7,000 rads.

91. The method of claim 82 wherein the incubating step (f) further comprises combining the CD8⁺ cells with the adherent cells at a ratio of about ten CD8⁺ cells to one adherent cell.

92. The method of claim 82 wherein the CD8⁺ cells are tested for at least one parameter selected from the group consisting of cytotoxic T cell activity, CTL cell purity, sterility and endotoxin content.

93. The method of claim 82 further comprising the step of introducing the CD8+ cells into a subject.

94. The method of claim 93 wherein CD4+ T lymphocytes comprising virus inhibiting nucleic acid are also introduced into the subject.

95. The method of claim 93 wherein CD34+ hematopoietic progenitor cells comprising virus inhibiting nucleic acid are also introduced into the subject.

96. The method of claim 93 wherein both CD34+ hematopoietic progenitor cells comprising virus inhibiting nucleic acid and CD4+ T lymphocytes comprising virus inhibiting nucleic acid are also introduced into the subject.

97. The method of claim 93 wherein IL-2 is administered to the subject following the cell introduction step.

98. The method of claim 82 wherein the subject tests positive for the presence of HIV antigen.

99. The method of claim 98 wherein antiretroviral therapy is stopped for a period of time following the introduction of the CD8+ cells into the subject.

100. A therapeutic cell product comprising a cytotoxic T-lymphocyte population primed for virus-specific CTL activity produced according to the method of claim 66.

101. A therapeutic cell product comprising a cytotoxic T-lymphocyte population transduced with virus-inhibiting nucleic acid and primed for virus-specific CTL activity produced according to the method of claim 82.

102. A method of treating a subject with an infectious disease, the method comprising administering to the subject a therapeutically effective dose of the therapeutic cell product of claim 101.

103. A method of treating a subject with an infectious disease, the method comprising:
(a) preparing non-naturally occurring antigen-presenting cells (nnAPC) which present at least one virus-specific antigen;
(b) harvesting a population of white blood cells from the subject;
(c) incubating a population of CD8+ cells obtained from the white blood cells in step (b) with the nnAPC cells;
(d) treating the CD8⁺ cells with one or more supportive cytokines; and
(e) introducing the CD8+ cells from step (d) into the subject.

104. The method of claim 103 wherein nnAPC cells present a plurality of the virus-specific antigens, and have been prepared by incubating the cells with at least two different peptides each comprising one of the virus-specific antigens, respectively.

105. The method of claim 103 further comprising incubating the CD8+ cells with non-proliferating peripheral blood mononuclear cell-derived adherent cells wherein the adherent cells present at least one of the same virus-specific antigenic peptides of step (a).

106. The method of claim 103 further comprising introducing at least one virus-inhibiting nucleic acid into the CD8+ cells, wherein the virus inhibiting nucleic acid is expressed in the lymphocytes.

107. The method of claim 106 wherein the virus-inhibiting nucleic acid is selected from the group consisting of transdominant proteins, intracellular antibodies, antisense molecules, RNA decoys, interfering RNAs, aptamers and ribozymes.

108. The method of claim 107 wherein the virus-inhibiting nucleic acid is a ribozyme.

109. The method of claim 103 wherein the virus-inhibiting nucleic acid is specific for a disease selected from the group consisting of Human papilloma virus, Cytomegalovirus, Epstein Barr Virus, Hepatitis A, Hepatitis B, Hepatitis C, Hepatitis D, Hepatitis E, Measles, Mumps, Polio, Rubella, Influenza, Yellow Fever, Japanese Encephalitis, Dengue, Rabies, Rotavirus, Varicella Zoster, Chikungunya Rift Valley Fever, Respiratory Syncitial Virus, Herpes Simplex, Coronaviruses, Marburg, Ebola, California Encephalitis Virus, JC Virus, Lymphocytic Choriomeningitis Virus, Parvovirus, Rhinovirus, Smallpox, HTLV-1, HTLV-2, and HIV.

110. The method of claim 109 wherein the virus-inhibiting nucleic acid is specific for HIV.

111. The method of claim 106 wherein the virus-inhibiting nucleic acid is passed to CTL progeny.

112. The method of claim 105 wherein the adherent cells are adherent monocytes obtained during the harvesting step (b).

113. The method of claim 112 wherein the adherent cells presenting at least one of the same virus-specific antigenic peptides of step (a) are produced by incubating the adherent cells with one or more different peptides, the or each peptide comprising one of the virus-specific antigens, respectively.

114. The method of claim 112 wherein the adherent monocytes are isolated from a suspension of peripheral blood monocytes after irradiating the suspension with a sufficient dose of γ-radiation necessary to prevent further cell proliferation of the peripheral blood monocytes.

115. The method of claim 114 wherein the dose of γ-radiation is in the range of about 3,000 to 7,000 rads.

116. The method of claim 105 wherein the incubating step (c) further comprises combining the CD8⁺ cells with the adherent peripheral blood monocytes at a ratio of about ten CD8⁺ cells to one adherent peripheral blood monocyte.

117. The method of claim 103 wherein the CD8⁺ cells are tested for at least one parameter selected from the group consisting of cytotoxic T cell activity, CTL cell purity, sterility and endotoxin content.

118. The method of claim 103 further comprising incubating a population of CD4+ T lymphocytes obtained from the white blood cells in step (b) with the nnAPC cells separately from the CD8⁺ cells, and introducing the CD4+ T lymphocytes into the subject.

119. The method of claim 118 further comprising adding one or more supportive cytokines to the CD4+ T lymphocytes prior to introducing the T lymphocytes into the subject.

120. The method of claim 118 further comprising introducing a virus-inhibiting nucleic acid into the population of CD4+ T lymphocytes prior to introducing the T lymphocytes into the subject, and wherein the virus inhibiting nucleic acid is expressed in the lymphocytes.

121. The method of claim 103 further comprising incubating a population of CD34+ haematopoietic progenitor cells with the nnAPC cells separately from the CD8+ cells for a period of time to stimulate the CD34⁺ cells prior to introducing the CD34⁺ cells into the subject.

122. The method of claim 121 further comprising adding one or more supportive cytokines to the CD34+ haematopoietic progenitor cells prior to introducing the CD34⁺ cells into the subject.

123. The method of claim 121 further comprising introducing a virus-inhibiting nucleic acid into the population of CD34+ haematopoietic progenitor cells prior to introducing the CD34⁺ cells into the subject, and wherein the virus inhibiting nucleic acid is expressed in the CD34⁺ cells.

124. The method of claim 103, wherein both CD34⁺ hematopoietic progenitor cells comprising virus inhibiting nucleic acid and CD4⁺ T lymphocytes comprising virus inhibiting nucleic acid are also introduced into the subject.

125. The method of claim 103 wherein the supportive cytokines are selected from the group consisting of IL-2, IL4, IL-7, IL-15 and IL-21.

126. The method of claim 125 wherein the supportive cytokines are IL-2 and IL-7.

127. The method of claim 103 wherein the CD8⁺ cells are incubated with the nnAPC cells for a period of from about 5 to 7 days.

128. The method of claim 103 wherein the supportive cytokines are added to the CD8+ cells about 4 days or more after step (c) is initiated.

129. The method of claim 103 wherein the subject has more than one infectious disease, and the nnAPC cells present at lest one virus-specific antigen for each disease, respectively.

130. The method of claim 103 wherein the nnAPC cells comprise a cell line.
